(19) United States
(12) Patent Application Publication
(10) Pub. No.: US 2026/0069448 A1
(43) Pub. Date: Mar. 12, 2026

(54) STOMA SITE PROTECTION DEVICES AND METHODS

(71) Applicant: THE CLEVELAND CLINIC FOUNDATION, Cleveland, OH (US)

(72) Inventors: Eric Yudelevich Blumrosen, Beachwood, OH (US); Andrew G. Williams, Mayfield Heights, OH (US); Shengqiang Gao, Beachwood, OH (US)

(21) Appl. No.: 19/388,438

(22) Filed: Nov. 13, 2025

Related U.S. Application Data
(63) Continuation of application No. 19/054,792, filed on Feb. 15, 2025.
Application No. 19/054,792 is a division of application No. 16/547,993, filed on Aug. 22, 2019, now abandoned.
(60) Provisional application No. 62/736,637, filed on Sep. 26, 2018.

(51) Int. Cl. A61F 5/44; A61F 5/445; A61F 13/05

(52) U.S. Cl. CPC A61F 5/4407; A61F 5/4408; A61F 5/445; A61F 13/05

(57) ABSTRACT

Stoma site protection devices, kits, assemblies and methods are provided that prevent or minimize leakage from a stoma. A stoma site protection device includes a conformal plug that can adapt to the stoma track. Kits and assemblies include additional components such as flexible external bumpers and bandages that provide multiple sealing of the stoma.

### CROSS-REFERENCE TO RELATED APPLICATIONS

This application is a continuation of U.S. patent application Ser. No. 19/054,792, filed Feb. 15, 2025, which is a divisional of U.S. patent application Ser. No. 16/547,993, filed Aug. 22, 2019, claims the benefit of U.S. Provisional Application No. 62/736,637, filed Sep. 26, 2018, entitled “STOMA LEAKAGE PREVENTION DEVICES, ASSEMBLIES AND KITS”. Each application is hereby incorporated by reference in its entirety for all purposes.

### TECHNICAL FIELD

The present disclosure relates to stoma site protection devices and methods that can include or be used with feeding tubes.

### BACKGROUND

Feeding tubes such as a jejunostomy tube (J-tube) and a gastrostomy tube (G-tube) may be used to provide nutritional support for users who are on prolonged artificial ventilation, have suffered central nervous system trauma or other condition in which the user is unable to maintain weight without assistance. The tubes are typically secured by an external retention ring on the outside skin surface and an internal retention feature, such as a small inflated balloon or bumper inside the stomach (G-tube) or small intestine (J-tube). The feeding tube may remain in place for several months either in a hospital or home setting.

External retention rings may slip, which can break the seal at the stomach/intestinal wall and allows acidic gastric fluid to leak onto the skin. This leakage can cause skin irritation and wounds that can progress to infection, requiring medical intervention or additional operations. Leakage may also occur around drainage tubes, such as chest tubes and surgical drains. These types of tubes generally remain in place for several days while the user is in the hospital. As such, a need exists for a device that addresses the problem of gastric fluid leakage as well as leakage of other fluids from tubes that are inserted in a patient.

### BRIEF DESCRIPTION OF THE DRAWINGS

FIG. 1 is a perspective view of a stoma site protection device according to an aspect of the present disclosure.

FIG. 2 is a cross-sectional view of the stoma site protection device of FIG. 1.

FIG. 3 is a perspective view of a stoma site protection device according to an aspect of the present disclosure.

FIG. 4 is a perspective view of a stoma site protection device and stoma tract patency device according to an aspect of the present disclosure.

FIG. 5 is a perspective view of a stoma site protection device according to an aspect of the present disclosure.

FIG. 6 is a perspective view of a stoma site protection device according to an aspect of the present disclosure.

FIG. 7 is a perspective view of a stoma site protection device in an un-expanded state according to an aspect of the present disclosure.

FIG. 8 is a perspective view of the stoma site protection device of FIG. 7 in an expanded state.

FIG. 9 is a perspective view of an external bumper of an assembly or kit according to an aspect of the present disclosure.

FIG. 10 is a perspective view of a bandage of an assembly or kit according to an aspect of the present disclosure.

FIG. 11 is a perspective view of a bandage of an assembly or kit according to an aspect of the present disclosure.

FIG. 12 is a perspective view of a bandage of an assembly or kit according to an aspect of the present disclosure.

FIG. 13 is a perspective view of a feeding tube assembly according to an aspect of the present disclosure.

FIG. 14 is a cross-sectional view of the feeding tube assembly of FIG. 13.

FIG. 15 is a cross-sectional view of an integrated medical device including a bandage, an external bumper and a stoma site protection device according to an aspect of the present disclosure.

FIG. 16 is a perspective view of an inner portion of a stoma site protection device according to an aspect of the present disclosure.

FIG. 17 is a cross-sectional view of a feeding tube assembly according to an aspect of the present disclosure.

FIG. 18 is an exploded view of a stoma site protection device and accompanying components and depicts an image of a stoma site protection device and accompanying components during a step of assembly according to an aspect of the present disclosure.

FIG. 19 depicts an image of a stoma site protection device and accompanying components during a step of assembly according to an aspect of the present disclosure.

FIG. 20 depicts an image of a stoma site protection device and accompanying components during a step of assembly according to an aspect of the present disclosure.

FIG. 21 depicts an image of a stoma site protection device and accompanying components during a step of assembly according to an aspect of the present disclosure.

FIG. 22 is a side view of a prior art feeding tube inserted in a user's stoma, stoma tract and stomach.

FIG. 23 depicts an image of a stoma site protection device and other assembled components during a step of insertion into a stoma, stoma tract, and stomach according to an aspect of the present disclosure.

FIG. 24 depicts an image of a stoma site protection device and other assembled components during a step of insertion into a stoma, stoma tract, and stomach according to an aspect of the present disclosure.

FIG. 25 depicts an image of a stoma site protection device and other assembled components during a step of insertion into a stoma, stoma tract, and stomach according to an aspect of the present disclosure.

FIG. 26 depicts an image of a stoma site protection device and other assembled components in their final position on a skin surface and in a stoma, stoma tract, and stomach according to an aspect of the present disclosure.

FIG. 27 is a cross-sectional view of the stoma site protection device and assembled components of FIG. 26.

### DETAILED DESCRIPTION

The present disclosure refers to the terms “upper,” “lower,” “top,” and “bottom” with respect to certain components. These terms refer to configuration of the components as illustrated in the drawings and as indicated by the character references. Further, as used herein with respect to a described element, the terms “a,” “an,” and “the” include at least one or more of the described element unless otherwise indicated. Further, the term “or” refers to “and/or” and “combinations thereof” unless otherwise indicated. In addition, when an element is referred to as being “over,” “on,” “attached” to, “connected” to, “coupled” to etc., another element, it can be directly over, on, attached to, connected to, coupled to, etc. the other element or intervening elements may also be present. In contrast, when an element is referred to as being, for example, “directly over,” “directly on,” “directly attached” to, “directly connected” to or “directly coupled” to another element, there are no intervening elements present. By “integral” or “integrated” is meant that the described components are fabricated as one piece or multiple pieces affixed during manufacturing or the described components are otherwise not separable using a normal amount of force without damaging the integrity (i.e. tearing) of the described components. A normal amount of force is the amount of force a user would use to remove a component meant to be separated from another component without damaging the components.

The present disclosure relates to stoma site protection device, kits, assemblies and methods of using a stoma site protection device to prevent leakages of fluid from a stoma. Disclosed devices, kits, assemblies, and methods include components, features, and/or steps that create a barrier to leakage at a stoma site. A stoma is an artificial opening made in a patient's skin to create a tract from an area inside the body to the patient's outer skin surface. A stoma is not a surgical incision site that is intended to be permanently closed after completion of the surgical procedure but rather an opening that is intended to remain open for a period of time after the stoma is created so that a percutaneous tube can be inserted through the stoma and remain in the patient for a period of time to deliver, remove, process, or otherwise accept fluid from the patient's body. A stoma can lead to a hollow organ or cavity such as organs of the gastrointestinal tract or the pleural cavity of the lungs. The below disclosure is described with respect to a feeding tube, which is a vessel through which fluids are delivered as well as through which fluids are drained from internal structures such as the stomach or the intestines via a stoma in the patient's body. However, devices, kits, assemblies and methods as described herein apply to other types of tubes that are inserted into an artificial opening made in a patient's body that extend from an exterior surface of the patient's body to an internal structure of the patient's body as well as other percutaneous tubes. All stoma site protection devices and accompanying components as described herein are used for medical purposes and are therefore sterile. As used herein, a “patient” or “user” is a mammal, such as a human being.

As stated above, leakage of bodily fluid, such as bile or gastric acid, can occur at the stoma site irritating skin around the stoma site. Such stoma site leakage currently has been described as being due to an incorrectly sized tube, a low water level in the internal retention element (such as a balloon), the stomach being too full of gastric acid or formula/food, and/or stomach gas. However, without wishing to be bound by theory, it is believed that a significant cause of leakage around the stoma site is, in actuality, the friction from the tube rubbing against the patient's skin at the stoma site during regular activity causing irritation and eventually creating a fissure between the skin and the end organ or cavity from which bile or gastric acid can leak. Basic bandages are not enough to keep tubes from moving which results in irritation and increased stoma size, resulting in leakage around the feeding tube. Daily activity, weight loss or gain, users tugging at insecure tubes, tube movement with contractions of the digestive system, for example, can cause tubes to move resulting in leakage. Such movement can break the seal at the stomach/intestinal wall and expand the diameter of the stoma tract through which the tube extends resulting in gastric/biliary fluid leakage onto the skin. This leakage can cause skin irritation and wounds that can progress to infection, cellulitis or abscesses. Such conditions can require medical intervention such emergency room visits, hospital admission, antibiotic use, or additional operations/tube exchange. Leakage may also occur around drainage tubes, such as chest tubes and surgical drains. Stoma site protection devices as disclosed herein can address this cause by allowing the tube to move freely without rubbing against the patient's skin. Such stoma site protection devices effectively act as bearings reducing the friction between the tube and the patient's skin.

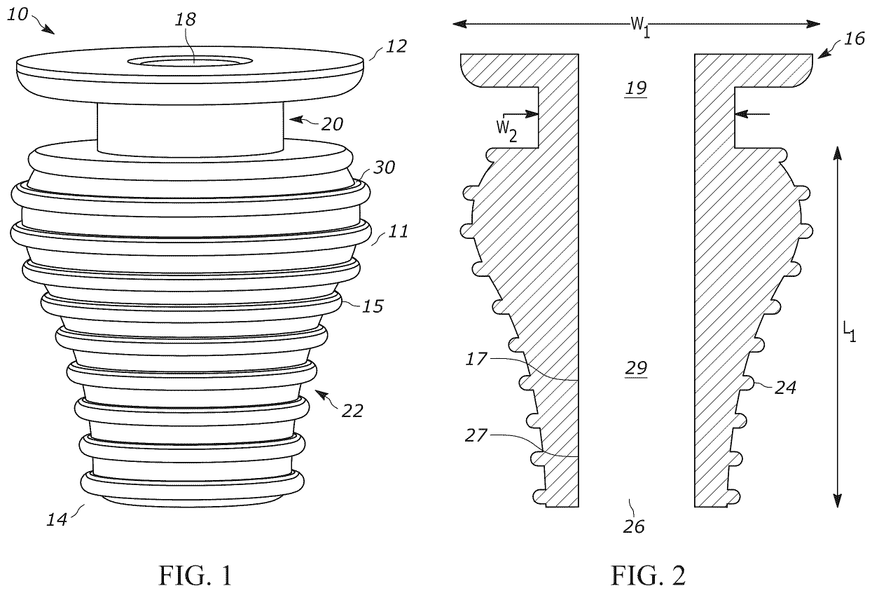

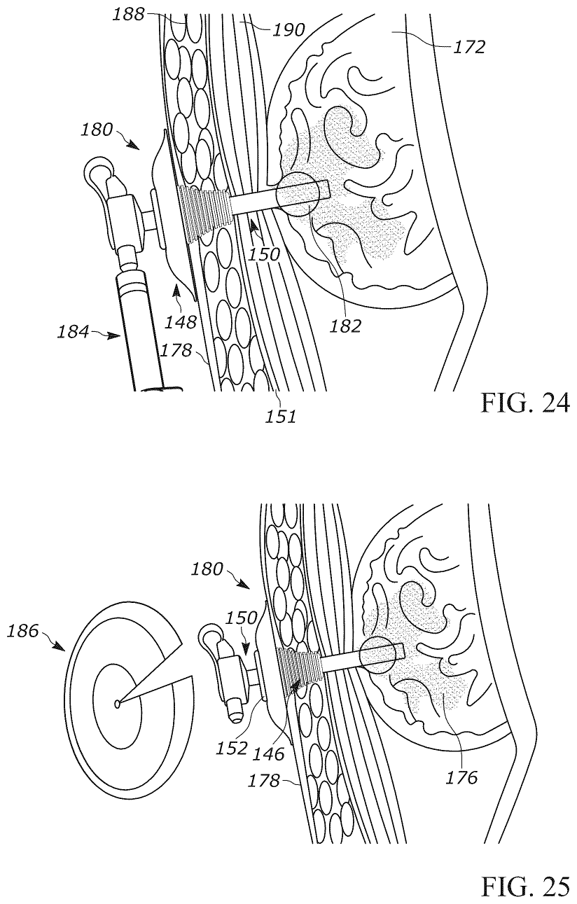

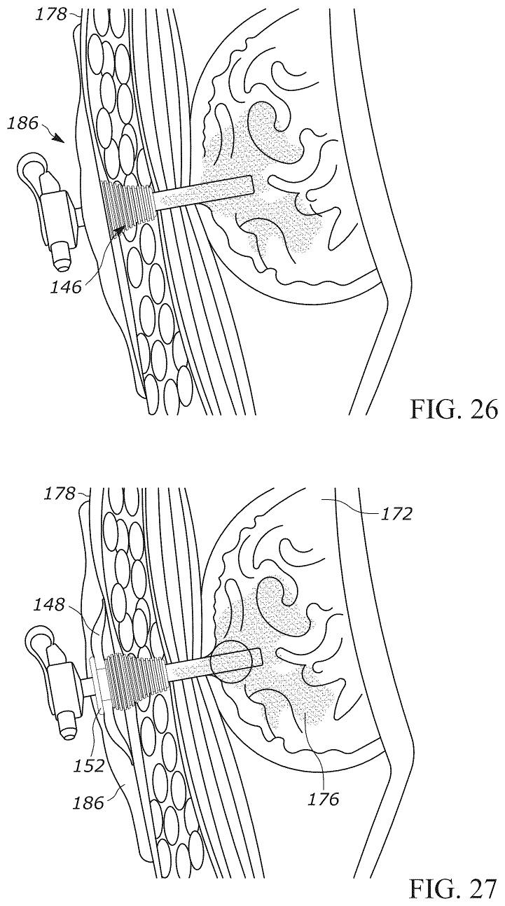

Referring to FIGS. 1 and 2, a stoma site protection device 10 can comprise a body 11 having a proximal end 12, a distal end 14, an outer surface 15, and an inner bearing surface 17. Body 11 can comprise a proximal collar 16 having a collar width W₁ and defining a top opening 18. An indented portion 20 can extend distally from proximal collar 16 and can have an indented portion width W₂ that is less than collar width W₁. W₁ of indented portion 20 can have a width that is substantially equal to the width of a stoma such that indented portion snugly conforms to and fits in stoma. The indented portion can be tubular in shape. As illustrated in FIG. 2, a lumen 19 can extend from top opening 18 to indented portion 20. Body 11 can also include a distal non-rigid or semi-rigid plug 22 extending distally from indented portion 20. Plug 22 can have a length L₁ that allows plug 22 to be positioned within the portion of the stoma tract that is the subcutaneous fat layer above the patient's muscle or fascia layer and below the patient's outer skin surface as depicted in FIGS. 24-27. Plug 22 can have an outer surface 24 and an inner bearing surface 27. Inner bearing surface 27 can define a plug lumen 29 and can define a bottom opening 26. The inner bearing surface can define a plug lumen having a diameter of between about 6 millimeters and about 10 millimeters in order to accommodate a percutaneous tube. Plug lumen 29 and bottom opening 26 can be axially aligned with and in fluid communication with lumen 19 and top opening 18 of proximal collar 16 to form a single fluid lumen. As shown in FIGS. 1 and 2, outer surface 24 of plug 22 can include ridges 30 about a circumference thereof. The ridges can extend partially or fully about the circumference of the outer wall of the plug. Such ridges can facilitate purchase between the outer wall of the plug and the stoma tract to provide a snug or tight fit of the plug in the stoma tract and prevent the plug from migrating or dislodging from the stoma tract.

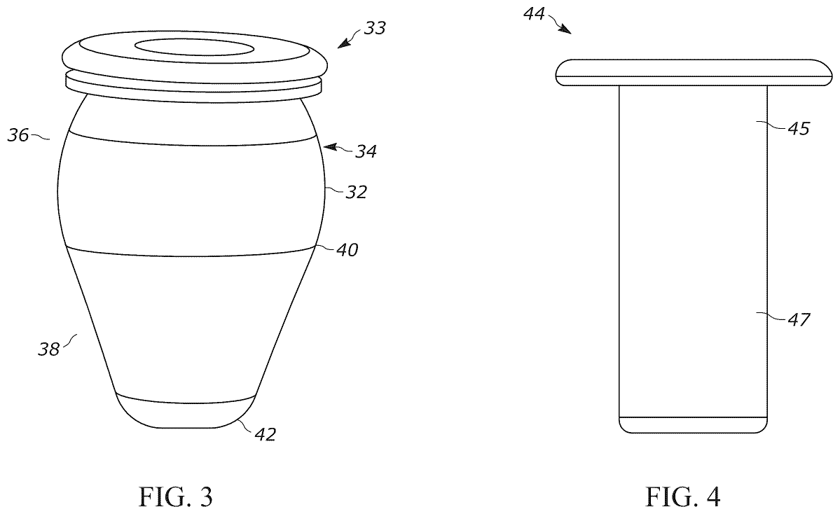

In other embodiments, as illustrated in FIG. 3, the outer surface 32 of a plug 34 of a stoma site protection device 33 can be smooth so as to minimize skin irritation in patients with sensitive skin, for example. Whether smooth or ridged, the plug can have a tapered or non-tapered shape. For example, referring to FIG. 3, the plug can have an upper portion 36 and a lower portion 38 with upper portion 36 having a bulbous shape and/or lower portion 38 tapering from a top end 40 to a bottom end 42 of lower portion 38. The bulbous shape of upper portion 36 can increase the surface area of plug 34 that contacts the proximal surface of the stoma track to further secure the plug in the stoma tract and minimize leakage from the stoma as well. The tapered lower portion can allow for a stoma tract having a smaller diameter, easier insertion of the plug, and easier longitudinal adjustment of the plug if necessary.

FIG. 4 illustrates a stoma site protection device 44 where indented portion 45 and plug 47 have substantially the same uniform diameter along the length of the indented portion and the plug. In this configuration, plug 47 has a substantially non-tapered shape (e.g. the diameter of the plug is substantially the same along the length of the plug). Such a configuration can be used while a stoma site is developing, for example. Once a feeding tube is placed, the stoma tract starts maturing in about one to about two weeks and is usually well formed in about four to about six weeks. Such a taperless configuration mimics the path of a maturing stoma. Once the stoma is matured and fully developed, a patient or caregiver can place a stoma site protection device having a tapered plug, for example, into the stoma for greater stability. Such a configuration can benefit temporary as well as chronic feeding tube patients.

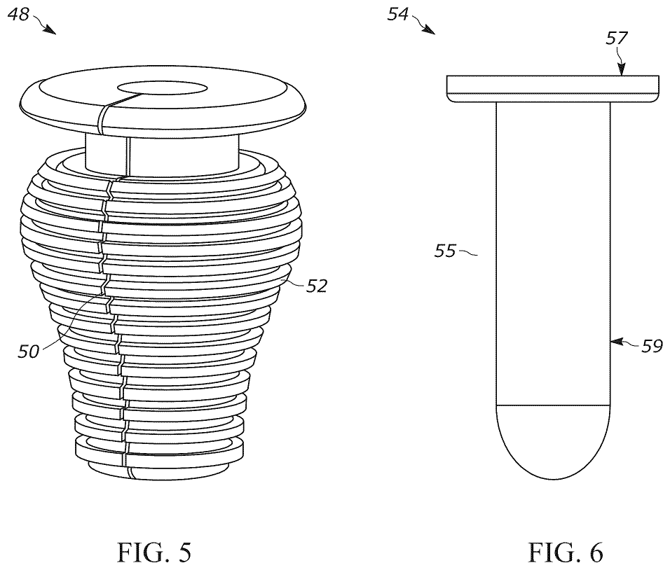

FIG. 5 illustrates a stoma site protection device 48 with a longitudinal slit 50 extending fully or partially along the outer wall 52 of the device. Such a slit allows the stoma site protection device to be placed around an existing percutaneous tube so that the tube does not need to be removed from the stoma. The device can be opened manually by a patient or caregiver, wrapped around the tube, and then closed. The end user can then slide the device distally along the tube into the stoma. Such a configuration can be useful for several different types of tubes, such as, for example, Dangler feeding tubes commonly used when developed a stoma site and other percutaneous tubes such as those used with ventricular assist devices (e.g. a left ventricular assist device (LVAD)).

FIG. 6 illustrates a non-cannulated stoma and stoma tract patency device 54 comprising a solid body 55 comprising a proximal collar 57 and a plug 59 extending distally from proximal collar 57. Stoma and stoma tract patency device 54 is solid with no lumen extending therethrough. Such a configuration can be used to maintain a stoma site when a patient does not have an internal retention element, such as a balloon, inserted in the stomach or other organ or cavity for some period of time. Because the device is solid in its entirety, it provides enough rigidity to prevent the site from closing but also reduces skin irritation.

The inner bearing surface of a plug of a stoma site protection device can have a Shore durometer hardness greater than the Shore durometer hardness of the outer wall so that the inner bearing wall is more rigid and the outer surface is more conformal. Such a configuration can allow for the feeding tube to have sufficient structural support within the plug such that the feeding tube stays in place but allows the plug to conform to the user's stoma tract, creating a stronger seal. In other words, the plug can be pliable enough to allow a feeding tube to move slightly as needed when the patient moves but still prevent leaks from occurring. The outer surface of a plug of a stoma site protection device can be fabricated from a semi-rigid material, such as a silicone material, so that the plug conforms to the stoma tract, adapts to varying stoma sizes, and achieves the appropriate fit and anchoring in the stoma tract. The stoma site protection device can be an integral, one-piece device.

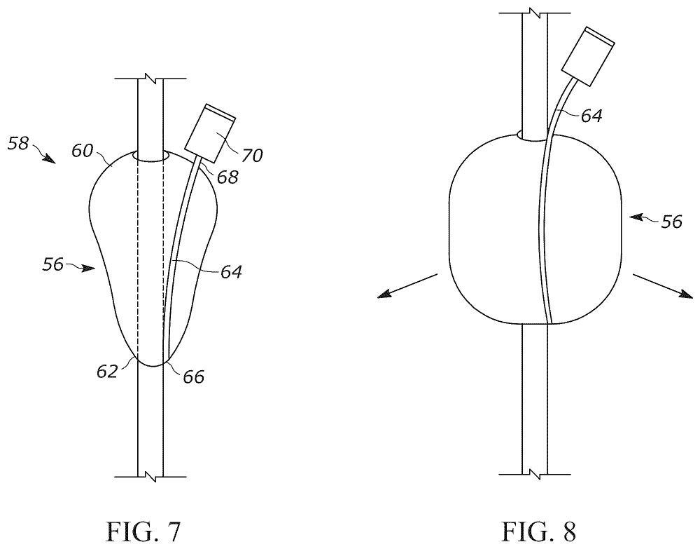

Referring to FIGS. 7 and 8, a plug 56 of a stoma site protection device 58 can be a balloon having a top portion 60 and a bottom portion 62 and that can transition from an un-inflated state to an inflated state. Plug 56 can have an inflation lumen 64 with one end 66 in fluid communication with bottom portion 62 of plug 56 and another end 68 in fluid communication with an infusion port 70 at top portion 60 of plug 56. Such a configuration allows the plug to be inflated upwards from the lowest point of the plug, thereby creating a seal as close as possible to the initial site of potential leakage in the digestive tract (such as the stomach or intestine), for example.

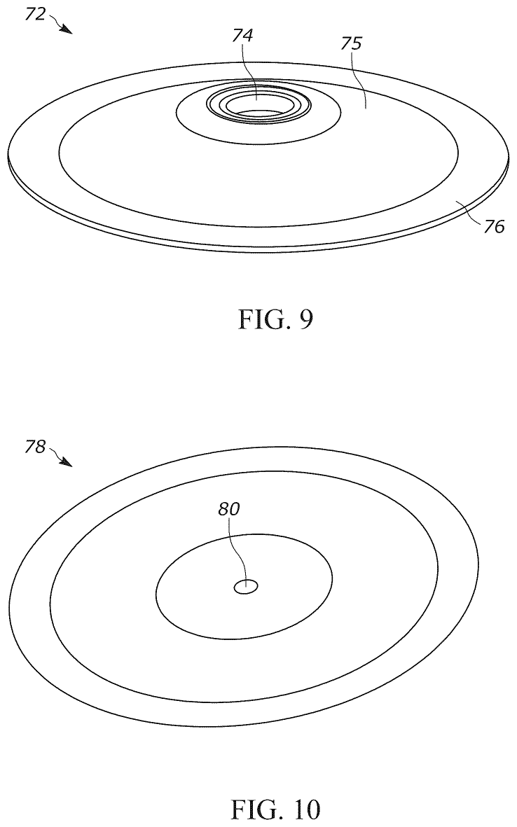

In certain aspects, a stoma site protection kit or assembly is provided that includes a stoma site protection device and other components that can improve the functionality of a feeding tube assembly or other medical tube assembly, such as by creating multiple seals of the stoma. For example, referring to FIG. 9, a stoma site protection assembly or kit can include a flexible bumper 72 that defines an opening 74 that can be axially aligned with the longitudinally extending lumen of a stoma site protection device in use and that is sized and configured to be placed about the indented portion of the body of a stoma site protection device as shown in FIGS. 21 and 27 and discussed in more detail below. As shown in FIG. 9, opening 74 can be surrounded by a dome shaped portion 75, which can be surrounded by a flange 76. In use, the bumper sits externally on the patient's skin preventing the stoma site protection device from slipping into the stoma and stabilizing the entire feeding tube assembly. For example, after the feeding tube is inserted in the patient, the bumper can be compressed over the stoma putting the entire tube assembly under tension preventing undesirable movement of the tube assembly. Dome shaped portion 75 ensures skin contact with the bumper is minimal and away from the stoma site (see e.g. FIG. 27) to prevent irritation at the stoma site. Skin contact can be limited to the area of the skin in contact with flange 76, which is spaced from the stoma site in use (see e.g. FIG. 27). The bumper can have an outer diameter of between about 59 millimeters and about 63 millimeters to adequately cover the stoma site as well as stabilize the percutaneous tube. The bumper can have a height of between about 5 millimeters and about 9 millimeters.

Referring to FIGS. 10-12, a stoma site protection kit or assembly can include a bandage 78 defining an opening 80 extending therethrough to accommodate the proximal end of a feeding tube. Bandage can secure a stoma site protection device and a bumper (in embodiments including a bumper) to the patient's skin.

A dressing such as a gauze dressing can be placed over the stoma to prevent leakage from the stoma and protect both the stoma and the feeding tube. Bandage 78 can cover the dressing and provide pressure to the stoma site protection device, the underlying dressing and the feeding tube. The bandage can be a flexible adhesive bandage and fabricated from silicone, for example. It can be taken off to change the gauze and access the feeding tube. The bandage can have a larger surface area than the bumper and can have an annular disc shape.

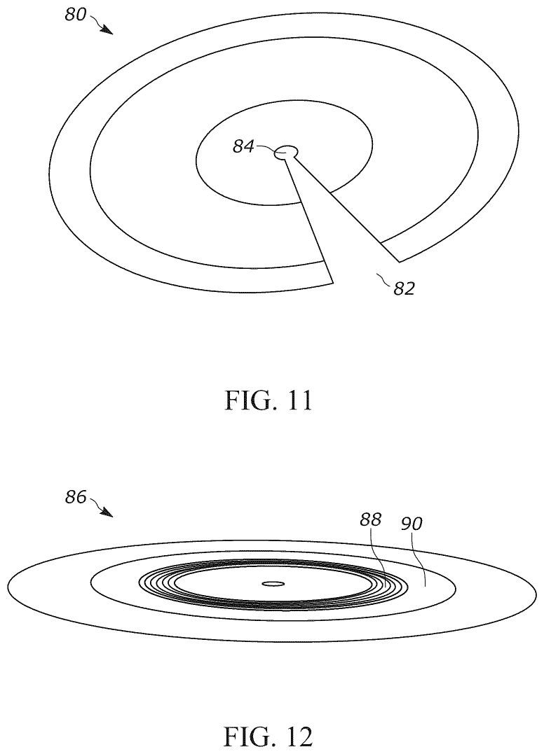

Referring to FIG. 11, in certain aspects, a bandage 80 can have a lateral slit 82 extending from and in fluid communication with a medial aperture 84. A slit can allow a patient or caregiver to place the bandage around the tube just below the proximal end or head of the tube. This allows for the smallest requisite aperture in the bandage and maximal coverage of the bandage over the bumper to assist with securing the device in place. By pulling apart the ends of the bandage defining the slit, a user can remove the bandage from the stoma site without having to remove the feeding tube. When replacing the bandage, the user can overlap the ends of the bandage defining the slit to securely adhere the bandage to the stoma site.

Referring to FIG. 12, bandage 86 can have a plurality of ribs 88 on top surface 90 thereof to provide sufficient points of contact for a final layer of an adhesive tape or covering to be positioned on the bandage. In particular, ribs 88 can provide enough surface area for proper adhesion of a final covering layer and can reduce tugging during removal of the final covering layer.

The bandage or other component that is placed over the stoma site protection device can include a moisture-sensitive, color-changing ink incorporated into the bandage or other protective component (such as an external bumper, for example) that changes color in the presence of liquid moisture. Incorporation of such an ink can alert a user or caretaker that fluid is leaking from the stoma. In the event that fluid, such as bile, does exit the stoma, the bandage can absorb such fluid before such fluid damages the stoma site. The bandage or other component that is placed over the stoma site protection device (such as an external bumper) can have a pH sensor to detect the presence of gastric acid, which could also indicate leakage from the stoma.

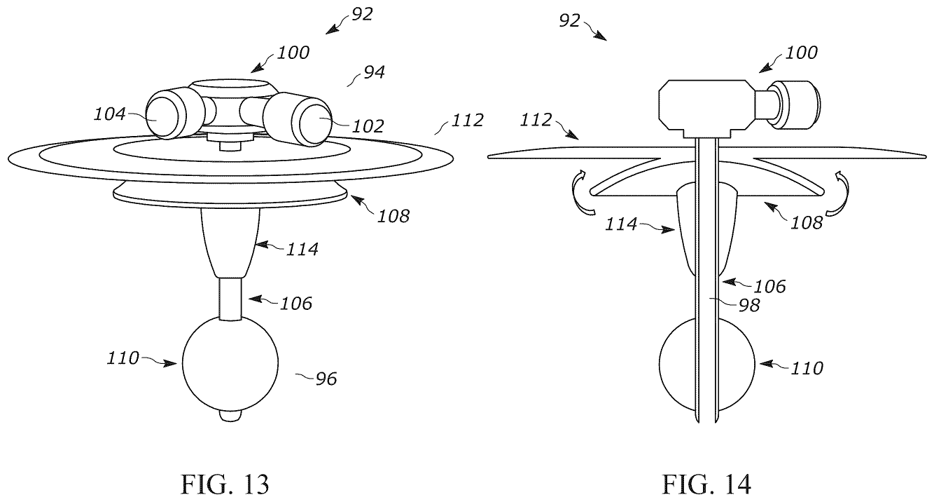

In certain embodiment, a feeding tube assembly is provided with a stoma site protection device and other optional components that can improve the functionality of the feeding tube. Referring to FIGS. 13-14, a feeding tube assembly 92 can comprise a proximal portion 94, a distal portion 96, and a lumen 98 extending longitudinally therebetween. Proximal portion 94 can comprise a proximal adapter 100 comprising one or more ports 102 and 104. Port 102 can be a patient or caregiver access port and can be longer than port 104 making it easier for the patient or caregiver to handle. Port 104 can be shorter than port 102 and designated for use by a doctor. A feeding tube 106 can extend distally from proximal adapter 100. A first bumper 108 can be disposed about tube 106 below proximal adapter 100 and a second bumper 110 can be disposed about tube 106 below first bumper 108 at distal portion 96. First bumper 108 can be dome shaped and flexible to facilitate compression of the bumper when a bandage 112 is applied over first bumper 108 (the arrows in FIG. 14 schematically illustrate the flexibility of first bumper 108. Second bumper 110 can be an expandable balloon or other structure that can positioned against the opening in the stomach or other internal structure. In use, the first bumper sits against the proximal end of the stoma tract on the outer surface of the user's skin and the second bumper sits on the distal end of the stoma tract on the inside surface of the stomach or intestines. Feeding tube assembly 92 can include a stoma site protection device comprising a non-rigid or semi-rigid plug 114 disposed about tube 106 between first bumper 108 and second bumper 110. The stoma site protection device can have a tapered configuration and can be inflated to plug and block the stoma opening. The feeding tube assembly can also include a flexible adhesive bandage 112 that is configured to be placed against the top surface of the first bumper. Bandage 112 can have a tapered design as shown in FIG. 14 to prevent or minimize snagging or interference with the patient's clothing, for example.

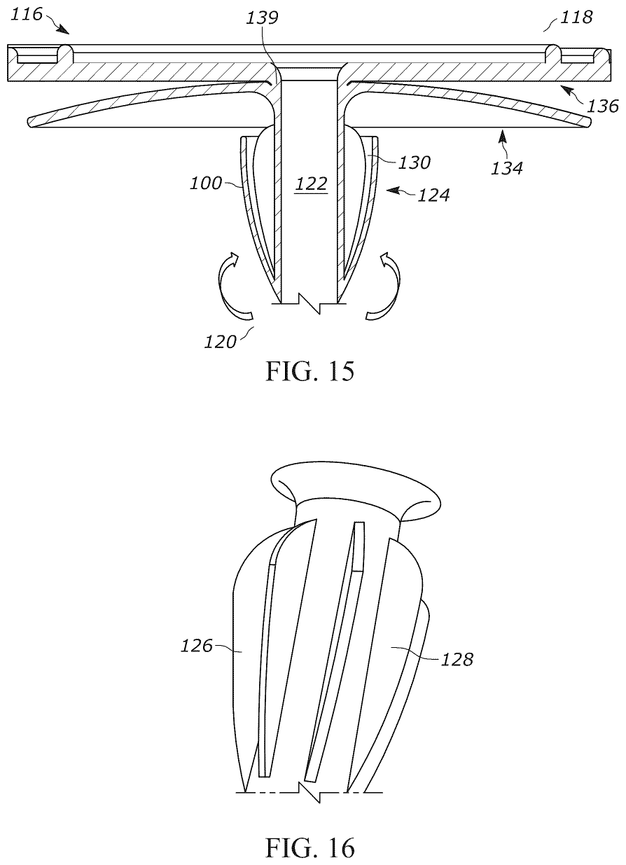

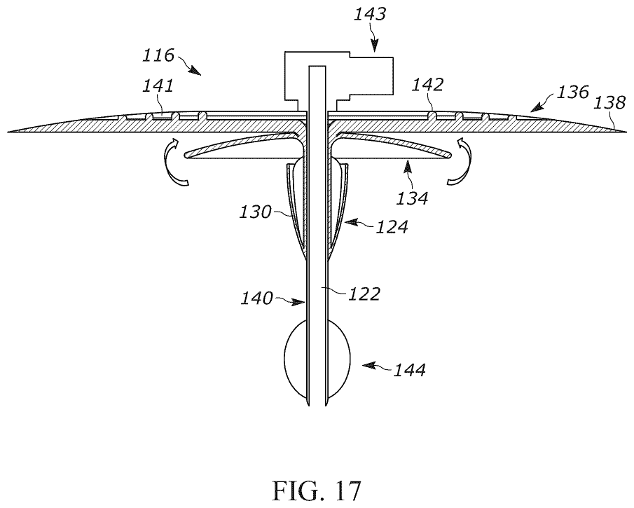

In certain embodiments, a stoma site protection device is part of an integrated single one-piece device that can plug the stoma tract and stabilize and secure a feeding tube assembly or other medical tube assembly. Referring to FIGS. 15-17, a device 116 can have a proximal portion 118, a distal portion 120 and a lumen 122 extending longitudinally therethrough. Distal portion 120 can comprise a stoma site protection device 124 that has an inner portion 126 with supporting flexible ribs 128 and is surrounded by a tapered cup 130 that has a flexible outer surface. During insertion into the stoma tract, the ribs can compress and then self-expand within the stoma tract as the taper cup inverts over the ribs as indicated by the arrows in FIG. 15. Device 116 can further comprise a proximal bumper 134 that is above and integral with stoma site protection device 124 and a bandage 136 that is above and integral with proximal bumper 134. Bumper 134 can be dome-shaped and flexible so that it is compressed as bandage 136 is applied against the user's skin (the arrows in FIG. 17 schematically illustrate the flexibility of proximal bumper 134. Bandage 136 can have a tapered surface 138 as shown in FIG. 17. Bandage 136 can have a tapered port 139 aligned with longitudinally extending lumen 122 of device 116 to aid in feeding tube ingress. FIG. 17 illustrates a feeding tube 140 inserted within longitudinally extending lumen 122 of device 116. The top surface 142 of bandage 136 can have a plurality of ribs 141 to provide sufficient points of contact for a final layer of an adhesive tape or covering to be positioned on the bandage. The points of contact also reduce the effort to remove the adhesive covering from the bandage and the user's skin. Device 116 can be part of a feeding tube assembly that includes feeding tube 140, a proximal adapter 143, and a distal bumper 144 as illustrated in FIG. 17.

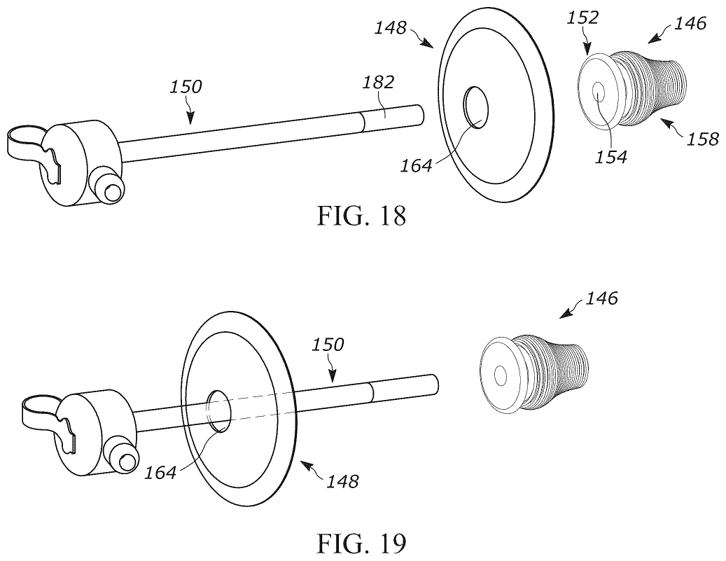

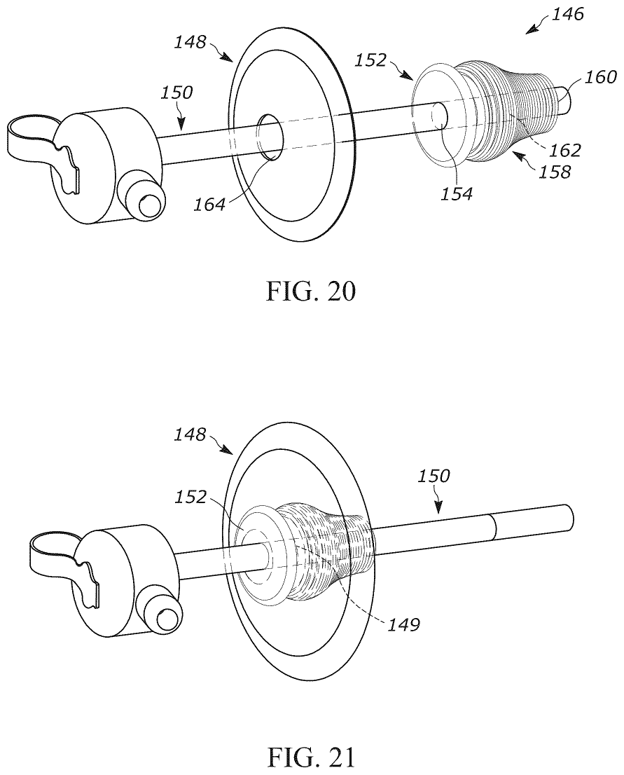

FIG. 18-21 depict images of a stoma site protection device and accompanying components during different steps of assembly with respect to a feeding tube, for example. FIG. 18 depicts a stoma site protection device 146 aligned with a bumper 148 and a feeding tube 150 having an internal retention element, such as a balloon 182 (shown inflated in FIGS. 24-27). Stoma site protection device can comprise a body having a proximal end, a distal end, an outer surface, an inner bearing surface, and a lumen extending longitudinally therethrough. The body can comprise a proximal collar 152 having a collar width and defining a top opening 154. An indented portion can extend distally from proximal collar 156 and can have an indented portion diameter that is less than the collar diameter. A plug 158 can extend distally from the indented portion and can have an inner bearing surface that defines a bottom opening 160 and a plug lumen 162. Plug lumen 162 can be axially aligned and in fluid communication with bottom opening 160 and top opening 154. Bumper 148 can be dome-shaped, for example, and can define a substantially central opening 164. Feeding tube 150 can be inserted into opening 164 of bumper 148, top opening 154 of collar 152, plug lumen 162, and through bottom opening 160 as shown in FIGS. 19 and 20. Because collar 152 is flexible, it can be urged through opening 164 of bumper 148 such that bumper 148 is positioned about indented portion 149 of stoma site protection device as shown in FIG. 21, which illustrates a fully assembled stoma site protection device 146, bumper, 148, and feeding tube 150.

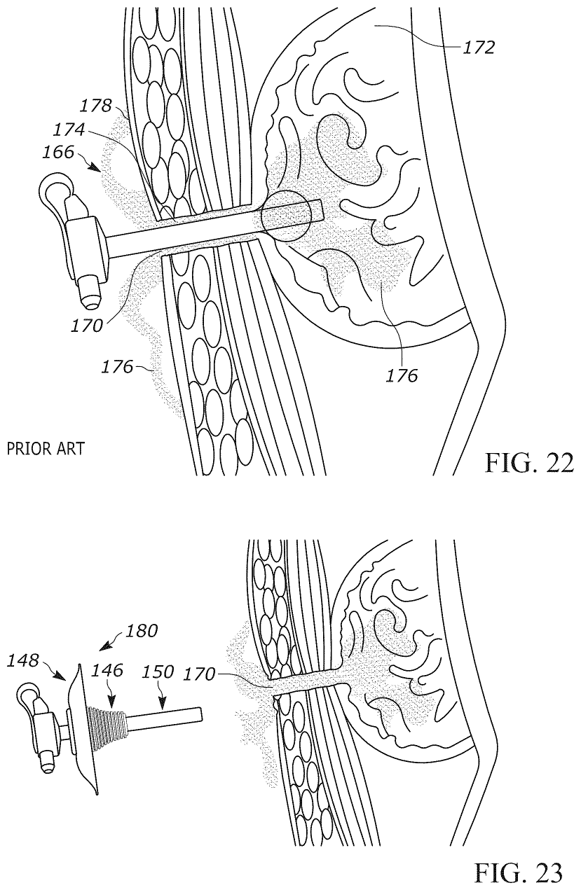

Methods of using a stoma site protection device are also provided herein. FIG. 22 illustrates a prior art feeding tube 166 inserted into a stoma 170 with the proximal end of the feeding tube at the stoma site and the distal end of feeding tube 166 in the stomach 172. As stated above, without wishing to be bound by theory, it is believed a significant cause of leakage around a stoma site is the friction from the tube rubbing against the skin at the stoma site during regular activity causing irritation and the break-down of skin eventually creating a fissure 174 (slightly exaggerated in FIG. 22 of the purposes of illustration) through which bile or gastric acid 176 can leak out of stoma 170 to the patient's skin outer surface 178. FIGS. 23-26 illustrate a method of using a stoma site protection device with a feeding tube to prevent or mitigate the leakage of fluid from stoma 170. Referring to FIG. 23, an assembled stoma site protection device 146, bumper 148, and feeding tube 150 (referred to herein with respect to FIGS. 23-27 as an “assembly 180” and as depicted in FIG. 21) is aligned with stoma 170. Referring to FIG. 24, stoma site protection device 146 and feeding tube 150 are inserted through stoma 170. Plug 158 is positioned in the subcutaneous fat layer 188 above the patient's muscle layer 190 (and/or fascia layer 151) and below skin outer surface 178, balloon 182 of feeding tube 150 is positioned in stomach 172, and bumper 148 is positioned over stoma 170. Bumper 148 sits externally on the patient's outer skin surface 178 and is compressed over stoma 170 putting the entire assembly under tension to prevent undesirable movement of assembly 180. The internal retention element of feeding tube 150 (illustrated in the presently described figures as a balloon 182) is inflated using a syringe 184, for example, to secure the distal end of feeding tube in stomach 172. As schematically illustrated in FIG. 25, because stoma site protection device 146 effectively acts as a bearing, there is no friction between the patient's skin outer surface 178 and feeding tube 150. As such, there is no break-down of skin 178 such that a fissure is created between the stomach and the stoma. With no such fissure, there is no leakage of bile or gastric acid 176 as is the case with prior art feeding tube 166 illustrated in FIG. 22. Referring to FIGS. 25-27, a bandage 186 is placed over collar 152 of stoma site protection device 146 and bumper 148 to secure stoma site protection device 146 and bumper 148 to the patient's skin.

As stated above, devices, kits, assemblies and methods as described herein can be used for medical tubes, such as percutaneous tubes, that are inserted into a stoma. Such tubes include feeding tubes that are placed through the nose, including nasogastric, nasoduodenal, and nasojejunal tubes; or placed directly into the abdomen, such as a gastrostomy, gastrojejunostomy, or jejunostomy feeding tube. Other medical tubes include chest tubes and surgical drains. Chest tubes include tubes used with ventricular assist devices including left and/or right ventricular assist devices. Non-limiting examples of dimensions of low profile feeding tubes is a length of between about 1.0 centimeters (cm) to about 4.5 cm and a tube diameter of between about 12 French (Fr) to about 24 Fr. Non-limiting examples of dimensions for adult chest tubes are about 20 Fr to about 40 Fr and about 6 Fr to about 26 Fr for children chest tubes. Non-limiting examples of dimensions for percutaneous drainage tubes are about 6.5 Fr to about 20 Fr.

Stoma site protection devices as disclosed herein prevent acid or other fluids from leaking out of the tube, provide pressure on the tube, and can be released from the tube in order to remove pressure or to allow drainage. The plug can have a depth such that it prevents acid or other fluid from accumulating near the upper end of the skin. As such, a stoma site protection device can prevent skin irritation surrounding the stoma. In addition, the plug does not have a depth so great that it compromises the seal between the feeding tube and the internal site, such as the stomach or intestine.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A feeding tube assembly comprising:
a proximal adapter comprising one or more ports;
a feeding tube extending distally from the proximal adapter;
a stoma site protection device distal to the proximal adapter comprising a balloon plug having a plug lumen through which the feeding tube extends and having an un-inflated state that transitions to an inflated state and comprising:
a top portion and a bottom portion; and
an inflation lumen with one end in fluid communication with the bottom portion and another end in fluid communication with an infusion port at the top portion; and
a balloon bumper disposed distal to the stoma site protection device and having an un-inflated state that transitions to an inflated state.

2. The feeding tube assembly of claim 1, wherein the proximal adapter, the feeding tube, the stoma site protection device and the balloon bumper are integrally connected.

3. A kit comprising the feeding tube assembly of claim 1 and further comprising:
another bumper codisposed about the feeding tube below the proximal adapter.

4. The kit of claim 3, wherein the other bumper is flexible and dome-shaped.

5. The kit of claim 3, further comprising an adhesive bandage defining an opening extending longitudinally therethrough and configured to be placed about the feeding tube distal to the proximal adapter.

6. The kit of claim 5, wherein the opening of the adhesive bandage is in fluid communication with a laterally extending slit.

7. The kit of claim 5, wherein the adhesive bandage has a top surface comprising a plurality of ribs.

8. The kit of claim 5, wherein the adhesive bandage has a moisture-sensitive, color changing ink incorporated therein.
